(19) United States
(12) Patent Application Publication
(10) Pub. No.: US 2023/0364288 A1
(43) Pub. Date: Nov. 16, 2023

(54) STORAGE ASSEMBLY HAVING AT LEAST ONE STORAGE COMPARTMENT

(71) Applicant: MERCEDES-BENZ GROUP AG, Stuttgart (DE)

(72) Inventors: Valentin MAIER, Ispringen (DE); Christoph MENZEL, Stuttgart (DE); Benno STAMP, Stuttgart (DE)

(21) Appl. No.: 18/029,736

(22) PCT Filed: Sep. 17, 2021
(86) PCT No.: PCT/EP2021/075562

§ 371 Date: Mar. 31, 2023

(30) Foreign Application Priority Data
Oct. 2, 2020 (DE) 10 2020 006 035.8

(51) Int. Cl. A61L 2/24; A61L 2/10

(52) U.S. Cl. CPC A61L 2/24; A61L 2/10; A61L 2202/11; A61L 2202/121; A61L 2202/122; A61L 2202/14

(57) ABSTRACT

A storage assembly has at least one storage compartment and a disinfection device, which has at least one light source that emits UV-C radiation and is integrated in the disinfection device. The storage compartment in which the disinfection device is integrated has a covering element and the storage compartment is radiopaque when the covering element is closed. The storage assembly includes a safety device, which detects a state of the covering element using detected signals when activating or deactivating the disinfection device.

### BACKGROUND AND SUMMARY OF THE INVENTION

Exemplary embodiments of the invention relate to a storage assembly having at least one storage compartment and a disinfection device, having at least one light source that emits UV-C radiation, integrated in this.

A charging and disinfection compartment for a vehicle is known from US 2015/0137747 A1. The charging and disinfection compartment comprises a wireless charging device for charging an electronic device and a lighting device. The lighting device comprises a disinfection device containing at least one photoluminescence indicator. A controller is connected to the wireless charging device and the lighting device and can control the photoluminescence indicator, while a sterilization process is undertaken with the disinfection device.

Exemplary embodiments of the invention provide a storage assembly having a storage compartment and a disinfection device that is integrated in this.

A storage assembly, in particular for a vehicle, has at least one storage compartment and a disinfection device that is integrated in this, having at least one light source that emits UV-C radiation. According to the invention, it is provided that the storage compartment in which the disinfection device is integrated has a covering element and the storage compartment is designed to be radiopaque when the covering element is closed. A safety device is additionally provided, which is in particular arranged on a housing of the disinfection device, and which is designed to detect a state of the covering element by means of detected signals when activating or deactivating the disinfection device.

A potential danger of a user of the disinfection device being blinded by the UV-C radiation, or of their skin coming into contact with the UV-C radiation can be considerably reduced by means of a storage assembly designed in this way. Firstly, the storage compartment having the disinfection device can be sealed in a radiopaque manner by means of the covering element and secondly, the safety device is provided, by means of which it can be ensured that the at least one light source that emits UV-C radiation is only then activated if it is detected that the covering element is closed. It is thus ensured as far as possible that UV-C irradiation of an object stored in the storage compartment, which can, for example, be a smartphone, a wallet or a key, only occurs when the storage compartment is closed, so that no UV-C radiation can leak out, i.e., out of the storage compartment. A degree of operating safety when using the disinfection device can be considerably increased by means of the storage assembly.

In one embodiment, the safety device is designed to activate the at least one light source that emits UV-C radiation when the covering element is closed and to deactivate it when the covering element is open. It can thus be ensured as far as possible that the at least one light source that emits UV-C radiation is then only activated if the covering element is closed. Additionally, the eyes and/or skin of a user of the disinfection device coming into contact with the emitted UV-C radiation can thus be substantially ruled out.

In a further embodiment of the storage assembly, the walls forming the storage compartment and/or an inner face of the covering element that faces the storage compartment are or is provided with a covering that reflects UV-C radiation. A degree of radiation efficiency for disinfecting the object stored in the storage compartment can thus be increased. For example, a necessary length of time for disinfecting the object can thereby be decreased.

In a development, the covering has at least one aluminum coating, by means of which the radiation efficiency of the UV-C radiation, which is emitted from the at least one light source of the disinfection device, is increased.

In an embodiment of the storage compartment arrangement, a pad that reflects UV-C radiation and/or a trough-shaped reflective receiving unit, having spacing elements that protrude from a base region at regular distances from each other, are or is arranged in the storage compartment. The object to be disinfected is arranged on the pad and/or in the receiving unit, wherein the object substantially rests on the spacing elements at certain points. The pad and/or receiving unit are or is designed to be reflective, so that an underside of the object to be disinfected, which is arranged on the spacing elements, can be simultaneously disinfected with an upper side of the object. A length of time for disinfecting the object can thereby be decreased again, so that it is possible to effectively disinfect the object in a comparatively short time.

A further embodiment of the storage assembly provides that at least the trough-shaped receiving unit and/or the pad are or is arranged in a framework element that can be taken out of the storage compartment. The disinfection device can thus in particular be integrated into the storage compartment as a retrofit option, wherein, to this end, a corresponding electrical connection for electrically connecting the at least one light source and the safety device to an electrical system of the vehicle is provided in the storage compartment.

In a further possible embodiment of the storage assembly, the storage compartment is arranged underneath a further storage compartment, wherein the covering element of the storage compartment at least partially forms a base element of the further storage compartment. The storage assembly thus has two storage compartments, of which the disinfection device is integrated into the lower storage compartment and the further upper storage compartment can be used for stowing further objects or already-disinfected objects.

In a possible embodiment, the further storage compartment has a further covering element for sealing the further storage compartment and the safety device is additionally designed to determine a state of the further covering element by means of detected signals. A safety design of the storage assembly therefore provides that the disinfection device can only then be activated if both the covering element of the storage compartment and also the further covering element of the further storage compartment are closed.

In one embodiment, the safety device has at least one sensor, in particular a lidar-based sensor, a RFID transponder, an infrared-based sensor, a Hall Effect sensor, and/or a capacitive sensor for determining the state of the respective covering element. The safety device in particular has a detection unit, which continuously detects signals, at least during operation of the vehicle, by means of which the state of the covering element and also of the further covering element can be reliably detected. It can then therefore be ensured as far as possible that the eyes and/or skin of a user come into contact with the UV-C radiation.

In a further embodiment, the covering element and/or the further covering element are or is designed as pivotable hinged lids or slide rollers. In particular at least the covering element of the storage compartment with the integrated disinfection device is designed in such a way that this storage compartment is radiopaque and thus no UV-C radiation leaks out.

### BRIEF DESCRIPTION OF THE DRAWING FIGURES

Exemplary embodiments of the invention are illustrated in greater detail below by means of drawings.

Here:

FIG. 1 schematically shows a perspective view of a section of a storage compartment with an integrated disinfection device,

FIG. 2 schematically shows a sectional view of a storage assembly with the storage compartment and a further storage compartment,

FIG. 3 schematically shows a sectional view of the storage assembly with the storage compartment and an open covering element and the further storage compartment and

FIG. 4 schematically shows a perspective view of the storage compartment with the integrated disinfection device and a partially transparent covering element.

Parts that correspond to one another are provided with the same reference numerals in all figures.

### DETAILED DESCRIPTION

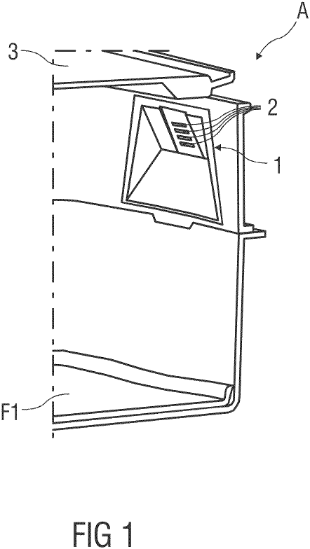

FIG. 1 shows a perspective view of a section of a storage compartment F1 with an integrated disinfection device 1 of a storage compartment assembly A.

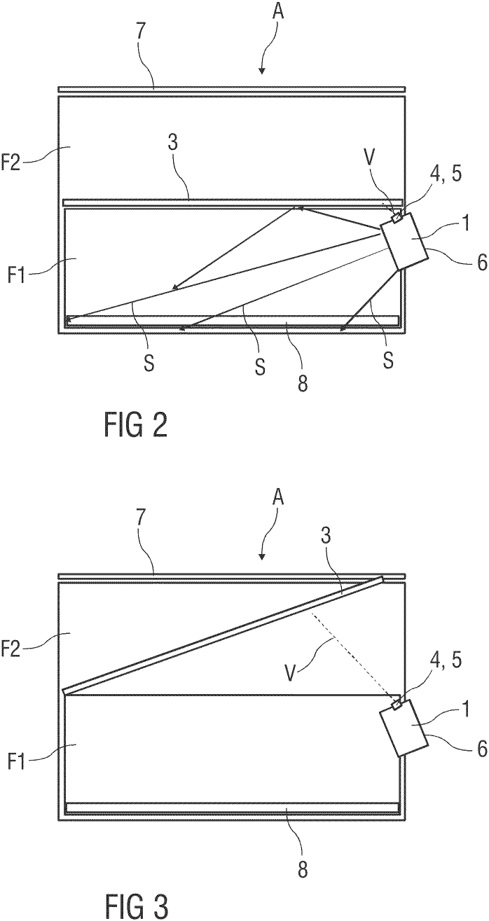

A sectional view of the storage assembly A with the storage compartment F1 and a further storage compartment F2 is shown in FIG. 2, wherein a sectional view of the storage assembly A with the storage compartment F1 and an open covering element 3 and the further storage compartment F2 is represented in FIG. 3.

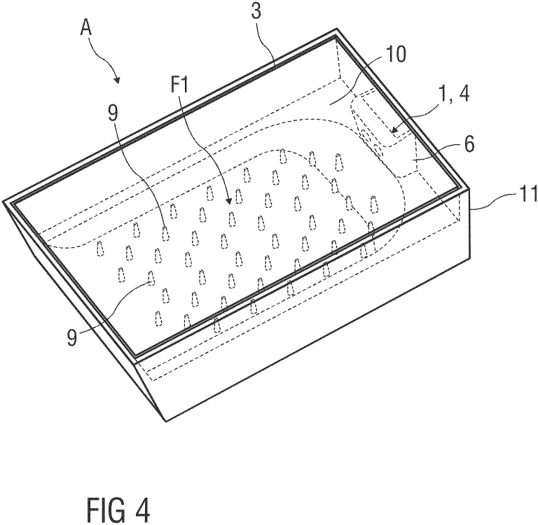

FIG. 4 shows a perspective view of the storage compartment F1 with the integrated disinfection device 1 and the partially transparent covering element 3.

The storage assembly A is arranged in a vehicle (not shown in more detail), wherein it can, for example, be a so-called cinematic compartment or a glove compartment. The storage assembly can also be integrated into a center console of the vehicle.

An object stored in the storage compartment F1, for example a smartphone, a key, a face mask, the storage compartment F1 itself, etc., can be disinfected by means of the disinfection device 1, in particular during the driving operation of the vehicle. To this end, the disinfection device 1 has an arrangement of light sources 2 that emit UV-C radiation S, in particular a so-called LED array. The number of the light sources 2 can vary, wherein the disinfection device has at least one light source 2, e.g., an LED.

It is possible to render bacteria and pathogens harmless by means of UV-C radiation S, whereby a risk of infection and disease, in particular for people, can be reduced. The short-wave UV-C radiation S is also comparatively dangerous for people, so eye and skin contact should be avoided. Due to the potential danger, it is necessary to be sure that irradiating an object stored in the storage compartment F1 is only carried out when the storage compartment F1 is closed, and no UV-C radiation S leaks out.

To this end, the storage compartment F1 can be sealed by means of the covering element 3, which, as is shown in FIG. 3, can be designed as a hinged lid. Alternatively, the covering element 3 can be designed as a slide roller and/or can have another suitable form. If the covering element 3 is closed, then the storage compartment F1 is designed to be radiopaque in relation to the UV-C radiation S, so that no UV-C radiation S leaks out. Because the storage compartment F1 is designed to be radiopaque, the UV-C radiation S can be comparatively safely contained. For example, a magnetic closure can be provided for the radiopaque closure of the storage compartment F1, wherein a magnet, in particular a permanent magnet or an electromagnet, is arranged in the covering element 3 and a ferromagnetic counterpart is arranged in a peripheral region of the storage compartment F1, wherein a position of the magnet corresponds with a position of the ferromagnetic counterpart.

In a possible embodiment, the covering element 3 has a so-called butterfly closure, so that an accidental opening of the covering element 3 can be ruled out as far as possible.

As is shown in FIGS. 2 and 3, the storage assembly A has a safety device 4, which is integrated into the disinfection device 1 in the exemplary embodiment in both Figures. In particular, the safety device 4 has at least one sensor 5, which, for example during active operation of the vehicle, continuously detects signals, by means of which a state of the covering element 3 is detected. It is in particular detected as the state of whether the covering element 3 is open or closed.

For example, a switch can be arranged as a sensor 5, wherein it can be detected, depending on a respective switch position, whether the storage compartment F1 is closed or open. Alternatively, or additionally, the state of the covering element 3 can be detected by means of detected signals of a lidar-based sensor 5, a transponder, an infrared-based sensor 5, a Hall Effect sensor and/or a capacitive sensor 5, as a component or components of the safety device 4.

In particular, the safety device 4 has a lidar-based sensor 5, the detection range of which is aligned in the direction of the covering element 3.

The sensor 5 is arranged on a housing 6 of the disinfection device 1, wherein detected signals of a control unit (not shown in more detail) are supplied for analysis and processing. In this exemplary embodiment, sensor 5 and light sources 2 are thereby integrated into a common module, i.e., into the disinfection device 1, which can also be jointly installed and substituted. It can furthermore make sense, depending on given installation space conditions, to arrange sensor 5 and light sources 2 separated from each other and/or at a distance from each other on the disinfection device 1.

For operating the disinfection device 1, it is provided that this is activated as soon as it is determined by means of detected signals of the sensor 5 that a distance between the sensor 4 and the covering element 3, detected by means of a waveform V, falls below a specified threshold value. A component tolerance is considered in relation to the specified threshold value, so that the disinfection device 1 is activated if the detected distance is in a distance range specified by means of the threshold value.

For safety reasons, the disinfection device 1 is then only activated if a specified length of time, e.g., of two seconds, is additionally exceeded, even if the interval falls below the specified threshold value,

The distance between the covering element 3 and the sensor 5 is determined in comparatively short intervals of time, so that an operating error of the disinfection device 1 can be ruled out as far as possible.

It can also be provided to this end that the disinfection device 1 is activated before the determining of the distance, without the respective light source 2 being switched on, and an irradiation of the stored object or of the stored objects with UV-C radiation S is only activated after checking the distance between the sensor 5 and the covering element 3.

In this above-described variant for determining whether the covering element 3 is closed or open, a previously defined distance range is necessary, which must be adapted depending on a respective environment.

In order to adapt the disinfection device 1 to further environmental conditions and/or storage compartments F1, F2, it is provided that the analysis is carried out without a defined, specific target value, i.e., threshold value. The disinfection device 1 is activated here as soon as no or a very small change in distance occurs over the time.

If the covering element 3 is closed, the determined distance between the covering element 3 and the sensor 5 in a time interval only changes slightly, for example due to vibration, so that the disinfection device 1 remains activated.

If it is detected that a change in distance exceeds a specified further threshold value during the time, then the disinfection device 1 is deactivated. The activation of the disinfection device 1 can only occur under the same conditions.

In particular, a distance range is specified within which the disinfection device 1 can usually be activated, for example when the determined distance between the covering element 3 and the sensor 5 is from 10 cm to 15 cm. Otherwise, there is the risk that a roof liner of the vehicle will be detected when the covering element 3 is open, where a small change in distance is also possible over the time. By means of the defined distance range, this operating error can be avoided as far as possible.

A combination of the further variants described above and a variant with the specific target value is also conceivable.

As well as a safety function, the sensor 5 can also be used as a switch element for activating and deactivating the disinfection device 1.

As soon as a vehicle user with an identifying feature lays a finger, for example an index finger, on the sensor 5 for a certain duration, for example for 2 seconds, and a specified, relatively low value, a value of 0 or an unmeasurable distance has previously been detected by means of detected signals, then the disinfection device 1 is activated without switching on the light source 2 that emits UV-C radiation S.

It is also possible to specify a certain gesture, e.g., by means of programming, to activate the disinfection device 1.

After the vehicle user has closed the covering element 3 and the corresponding spacing of the covering element 3 in relation to the sensor 5 has been detected by means of the safety device 4, then the light sources 2 are activated and the irradiation, i.e., a disinfection process of an object stored in the storage compartment F, is started.

An activation process of the disinfection device 1 can, for example, be confirmed by the vehicle user by means of a finger on sensor 5, detected approach to the sensor 5 and/or by means of certain gestures, depending on the closed state of the storage compartment F1 determined by means of the safety device 4, by means of a light emitting diode that is arranged visibly on the storage compartment F1 for the vehicle user and/or by means of haptic feedback, e.g. to the disinfection device 1 and/or the storage compartment F1.

As is shown in FIGS. 2 and 3, the storage assembly A comprises two storage compartments F1, F2 according to the embodiment shown in FIGS. 2 and 3, wherein a further storage compartment F2 is arranged above the storage compartment F1 with the integrated disinfection device 1.

The covering element 3 of the storage compartment F1 at least partially forms a base element of the further storage compartment F2.

If a vehicle user or another occupant of the vehicle intends to disinfect an object, it is initially necessary to open a further covering element 7 of the further storage compartment F2. The covering element 3 of the storage compartment F1 located in the further storage compartment F2 is subsequently opened, in order to store the object to be disinfected in the storage compartment F1 for the irradiation with the UV-C radiation S.

To this end, it is provided that the disinfection device 1 for disinfecting the object in the storage compartment F1 is only then activated if it is detected by means of detected signals of the safety device 4 that both the covering element 3 attached to the storage compartment F1 and the further covering element 7 attached to the further storage compartment F2 is closed. It can also be provided that the safety device 4 has several, in particular two, sensors 5 for detecting the respective state of the two covering elements 3, 7. The above-mentioned detection units or sensors 5 can be used in order to detect signals, by means of which the respective state of the covering elements 3, 7 can be determined. Using two sensors 5, the state of the covering element 3 of the storage compartment F1 is detected by means of detected signals of a sensor 5 and the state of the further covering element 7 of the further storage compartment F2 is detected by means of detected signals of a further sensor that is not shown in more detail. Safety in relation to the detection of the states of the covering elements 3, 7 for a safe start-up of the disinfection device 1 can thereby be increased.

According to the embodiment shown in FIGS. 2 and 3, the covering element 3 of the storage compartment F1 with the integrated disinfection device 1 is designed as a hinged lid, wherein the safety device 4 is correspondingly arranged in order to detect the state of the covering element 3 and of the further covering element 7.

The covering element 3 is provided with a reflective covering, in particular a coating, for example aluminum, on its interior face, so that the UV-C radiation S directed upwards is reflected by the covering element 3, in particular its coating, when the disinfection device 1 is activated, in order to increase radiation efficiency for disinfecting the object stored in the storage compartment F1.

Additionally, or alternatively, the walls forming the storage compartment F1 can be provided with a reflective covering, in particular a coating, in order to increase the radiation efficiency for disinfecting the object stored in the storage compartment F1.

Additionally, a pad 8 that reflects the UV-C radiation S is arranged on a base region of the storage compartment F1, in order to increase the radiation efficiency. In particular, the pad 8 has spacing elements 9 shown as an example in FIG. 4, which are arranged at regular distances from each other. An object to be disinfected then lies on the spacing elements 9, so that an underside of the object to be disinfected is also irradiated by means of the UV-C radiation S reflected by means of the reflective pad 8. An efficiency of the disinfection device can thereby be increased and a necessary time for disinfection can be decreased.

If the covering element 3 of the storage compartment F1 is closed, the further storage compartment F2 can be used for stowing further objects, for example belonging to the vehicle user or another occupant. A disinfection process in the storage compartment F1 is not affected by this.

In an exemplary embodiment shown in FIG. 4, which can represent a retrofit option for a vehicle, a trough-shaped receiving unit 10 with spacing elements 9 is provided, which can be arranged in a storage compartment arrangement A for forming the storage compartment F1 with integrated disinfection device 1. A removable framework element 11 is also provided, in which the trough-shaped receiving unit 10 can be inserted. For example, the above-described reflective pad 8 can be formed as a trough-shaped receiving unit 10.

The framework element 11, the receiving unit 10,and the covering element 3 here form a retrospectively formable storage compartment F1. For example, the framework element 11 is formed as a disinfection housing with covering element 3 and the trough-shaped receiving unit 10 is at least fixed in a form-fitting manner, in particular clipped onto the framework element 11, so the disinfection housing.

For example, the covering element 3 and the receiving unit 10, e.g., in the form of the reflective pad 8, can be inserted anywhere; only the framework element 11 has to be adapted vehicle-specifically. Cost savings in relation to a retrospective integration of the disinfection device 1 in a storage compartment F1 can be achieved from this.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

1-10. (canceled)

11. A storage assembly, comprising:
a storage compartment comprising a covering element, wherein the storage compartment is configured to be radiopaque when the covering element is closed;
a disinfection device integrated into the storage compartment, wherein the disinfection device comprises at least one light source configured to emit UV-C radiation; and
a safety device configured to detect, using detected signals, a state of the covering element to activate or deactivate the disinfection device.

12. The storage assembly of claim 11, wherein the safety device is configured to activate the at least one light source when the covering element is closed and to deactivate it when the covering element is open.

13. The storage assembly of claim 11, wherein the storage compartment includes walls and the covering element includes an inner face that faces the storage compartment, wherein the walls of the storage compartment or the inner face of the covering element includes a covering that reflects the UV-C radiation.

14. The storage assembly of claim 13, wherein the covering that reflects the UV-C radiation includes at least one aluminum coating.

15. The storage assembly of claim 11, wherein at least one of the following is arranged in the storage compartment:
a pad configured to reflect the UV-C radiation; or
a trough-shaped reflective receiving unit having spacing elements protruding from a base region at regular distances from each other.

16. The storage assembly of claim 15, wherein the reflective pad or the trough-shaped receiving unit is arranged in a framework element configured to be removeable from the storage compartment.

17. The storage assembly of claim 11, wherein the storage compartment is arranged underneath a further storage compartment, wherein the covering element of the storage compartment at least partially forms a base element of the further storage compartment.

18. The storage assembly of claim 17, wherein the further storage compartment has a further covering element configured to seal the further storage compartment, and wherein the safety device is configured to determine a state of the further covering element using detected signals.

19. The storage assembly of claim 11, wherein the safety device includes a lidar-based sensor, an RFID transponder, an infrared-based sensor, a Hall Effect sensor, or a capacitive sensor to determine the state of the covering element.

20. The storage assembly of claim 18, wherein the covering element or the further covering element is a pivotable hinged lid or slide roller.
